(19) United States
(12) Patent Application Publication
(10) Pub. No.: US 2008/0261858 A1
(43) Pub. Date: Oct. 23, 2008

(54) Fragrance Compositions

(75) Inventors: David O'Halloran, Milltown, NJ (US); Alfredo Vinci, Pennington, NJ (US)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 12/090,297

(22) PCT Filed: Oct. 12, 2006
(86) PCT No.: PCT/CH06/00566

§ 371 Date: Jun. 27, 2008

Related U.S. Application Data
(60) Provisional application No. 60/727,757, filed on Oct. 18, 2005.

(51) Int. Cl. A61K 8/33; A61Q 13/00

(52) U.S. Cl. 512/4; 512/11

(57) ABSTRACT

An eau de Cologne composition that comprises a water-soluble polyurethane and a low molecular weight water-soluble saccharide. Preferably the polyurethane is Polyurethane-1 and the saccharide sucralose. A dried film of the composition on the skin releases a burst of fragrance when wetted, for example by licking or kissing, and in addition tastes pleasant and does not become sticky.

This invention relates to fragrance compositions and more particularly to eau de Cologne-type fragrance compositions that emit fragrance when moistened.

Most fragrance compositions, including water-ethanol-based eau de Cologne-type preparations, are not intended to be moistened. They are simply applied to the skin and the odour lasts while there is still composition to evaporate. However, there is a current interest in such preparations that will have an increased emission when wetted, for example, by licking or kissing. It has been found that conventional controlled release technologies, such as the various types of encapsulation, simply do not work satisfactorily in such an application.

It has now been found that a particular composition will exhibit this desirable characteristic. The invention therefore provides an eau de Cologne composition that comprises a water-soluble polyurethane and a low molecular weight water-soluble saccharide.

The invention further provides a method of providing to a dried eau de Cologne composition the ability to provide a boost of fragrance when wetted, comprising the inclusion in the composition of a water-soluble polyurethane and a low molecular weight water-soluble saccharide.

By “eau de Cologne composition” is meant a solution of fragrance ingredients in a water-ethanol mixture. Eaux de Cologne are very well known formulations in the art of cosmetics and perfumery and typically comprise 10-90% by wt ethanol, 1-90% by wt water, 1-50% by wt fragrance or fragrance material with typical weight ratios of ethanol:water:fragrance being 8:1:1. Fragrance compounds used include essential oils derived from plants such as oil of sandalwood, peppermint oil, rose absolute or eau de rose, patchouli oil and ginger root oil. Synthetic fragrance compounds and mixtures thereof may also be used.

The use of polymers such as PVP, PVA, cellulosics, polyols and various other high molecular weight polymers is well known. Such polymers have good adhesion to the skin and will hold the fragrance thereon. However, such polymers often have a poor spraying effect and create a less desirable film on the skin, which film will not be as comfortable to wear. In contrast, the use of polyurethane in combination with polysaccharides, combined with a fragrance oil in a hydro-alcoholic solution and sprayed on the skin, leaves a smooth, silky, comfortable film which “entraps” the fragrance in the film only releasing it when the film is made moist or wet.

The safety of polyurethanes is well known and is documented in literature. The material is widely used in biomedical applications and cosmetics. Polyurethane is an inert non-reactive material and it is generally recognized as safe.

The polyurethane may be selected from one or more of the water-soluble polyurethanes (PUs) known to the art. Typical suitable PUs will have a weight-average molecular weight of from 10,000-15,000. Particularly useful polyurethanes are those commonly used in hair sprays. Typical of the commercial materials available is Luvise™ P.U.R. of BASF A.G., known to the art as “Polyurethane-1” (see “International Cosmetic Ingredient Dictionary and Handbook”, (Wenninger, Canterbery, McEwen, Eds.) 8^{th} Ed. (2000), Cosmetic, Toiletry and Fragrance Association).

Polyurethane-1, although preferred from film texture and skin feel points of view, can be replaced with higher molecular weight grades of polyurethane, such as Polyurethanes-2, -3 or -4. However, the higher molecular weight materials will require different processing and may give uncomfortable skin feel and have higher odor values. They are also less resistant to flaking and are at their most effective when plasticized.

It is possible to use blends of polyurethanes. However, it is preferred that, in any such blend, Polyurethane-1 shall comprise at least 70%, more preferably 80%, even more preferably 90% and most preferably 98% by weight of any such blend. As hereinabove mentioned, it is preferred that Polyurethane-1 be the sole polyurethane present.

The saccharide may be selected from one or more of the low molecular weight, water-soluble saccharides known to the art. By “low molecular weight” is meant a saccharide that has no more than three saccharin ring units. Preferred polysaccharides have a molecular weight of 600 maximum. Typical of such materials that are useful in this invention are monosaccharides such as dextrose and disaccharides such as sucrose. Saccharides according to this invention may be natural materials, or they may be synthetically altered in structure.

The low molecular weight saccharides hereinabove mentioned have the additional property of being sweet-tasting. This is especially advantageous when the moisture releasing the fragrance originates from the mouth—a bitter- or bad-tasting composition is not desirable. However, the use of many of the more common polysaccharides such as sucrose, dextrose, maltose results in a less desirable skin feel and stickiness, will create a less desirable film on the skin and will not be as comfortable to wear. This can be countered to some extent by adding to the composition water-soluble emollients such as propoxylated or ethoxylated branched or straight chain alcohols or fatty acids having at least 8 carbon atoms and an average number of ethylene oxide and or propylene oxide units of from 2-20. Other materials such as simethicone or dimethicone polyols may also be used.

However, in a particularly useful embodiment of the invention, there is used a saccharide that avoids completely the problem of stickiness. The compound is sucralose, an artificial sweetener derived synthetically from sucrose, and having the chemical name 1,6-dichloro-1,6-dideoxy-β-D-fructofuranosyl-4-chloro-4-deoxy-alpha-D-galactopyranoside (CAS number 56038-13-2). Surprisingly, it has been found that the use of sucralose gives a composition that not only is particular efficacious in releasing fragrance when moistened, but that also has a reduced tendency to stickiness after moistening.

Although sucralose is the preferred material for use in this invention and it preferably comprises 100% of the saccharide, it is possible to use it blended with other saccharides. In such a case, sucralose shall comprise at least 70%, more preferably 80%, even more preferably 90% and most preferably 98% by weight of any such blend.

In addition to the ingredients described hereinabove, the compositions of this invention can also comprise art-recognised ingredients added in known quantities to enhance particular properties. Examples of such materials include biological materials such as aloe vera, chamomile or any cosmetic botanical material. Other possible additives include vitamins, colorants such as FD&C or D&C organic dyes or natural colorants such as carmine red, titanated micas or inorganic pigments such as iron oxides or titanium dioxide or other pearlizing agents. This list is not intended to be exhaustive, and it is well within the skill of the art to use other ingredients.

The compositions of the invention have the advantages that, when applied to the skin and dried, a wetting of the resultant dry film, for example, by licking or kissing releases a burst of fragrance. In addition, the licker or kisser receives a sweet taste, and the wetted film dries and does not leave a sticky residue. The invention therefore provides a method of providing a fragrancing effect, comprising the steps of: providing a composition as hereinabove defined on a human body, permitting said composition to dry upon the body, and subsequently, upon wetting of the composition, releasing a fragrance therefrom.

The invention is further described with reference to the following non-limiting examples

### EXAMPLES

#1 Wet Formulation

| Ingredient | % Wt |
|---|---|
| Fragrance | 15.0 |
| Polyurethane - 1 | 1..0 |
| Sucralose | 2.0 |
| Denatured Ethanol | 78.0 |
| Water | 4.0 |

#2 Wet Formulation

| Ingredient | % Wt |
|---|---|
| Fragrance | 15.0 |
| Polyurethane - 1 | 1.0 |
| Sucralose | 2.0 |
| d,l-panthenol | 0.5 |
| Denatured Ethanol | 78.0 |
| Water | 3.5 |

#3 Wet Formulation

| Ingredient | % Wt |
|---|---|
| Fragrance | 12.0 |
| Polyurethane - 1 | 1.0 |
| Sucralose | 2.0 |
| d,l-panthenol | 0.5 |
| Denatured Ethanol | 78.0 |
| Water | 6.5 |

### Testing:

5 individuals evaluated the reboosting and edible capabilities of the compositions given above.

After an initial application (spray) of the fragrance and a dry-out period of about 30 minutes each individual noticed a strong upsurge (reboost) of the fragrance after wetting the polyurethane film and also a very nice sweet taste to the palate caused by the sucralose. The invention could be used not only as a reboosting composition but also as an edible GRAS composition when prepared with edible fragrances and/or flavors.

1. An eau de Cologne composition which comprises a water-soluble polyurethane and a low molecular weight water-soluble saccharide.

2. A composition according to claim 1, in which the polyurethane has a weight-average molecular weight of from 10,000-15,000.

3. A composition according to claim 1, in which the polyurethane is Polyurethane-1.

4. A composition according to claim 1, in which the saccharide is sucralose.

5. A composition according to claim 4, in which the polyurethane is Polyurethane-1.

6. A method of providing to a dried eau de Cologne composition the ability to provide a boost of fragrance when wetted, comprising the inclusion in the composition of a water-soluble polyurethane and a low molecular weight water-soluble saccharide.

7. A method according to claim 6, in which the polyurethane has a weight-average molecular weight of from 10,000-15,000.

8. A method according to claim 6, in which the polyurethane is Polyurethane-1.

9. A method according to claim 6, in which the saccharide is sucralose.

10. A method according to claim 9, in which the polyurethane is Polyurethane-1.

11. A method of providing a fragrancing effect, comprising the steps of:
providing a composition according to claim 1 on a human body,
permitting said composition to dry upon the body, and
subsequently, upon wetting of the composition, releasing a fragrance therefrom.
